(19) United States
(12) Patent Application Publication
(10) Pub. No.: US 2006/0269630 A1
(43) Pub. Date: Nov. 30, 2006

(54) Morinda citrifolia as a 5-Lipoxygenase inhibitor

(76) Inventors: Afa Kehaati Palu, Orem, UT (US); Brett West, Orem, UT (US); Claude Jarakae Jensen, Cedar Hills, UT (US); Chen Su, West Jordan, UT (US); Bing-Nan Zhou, Sandy, UT (US); Stephen P. Story, Alpine, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/500,728

(22) Filed: Aug. 8, 2006

Related U.S. Application Data
(62) Division of application No. 10/417,406, filed on Apr. 16, 2003.

(51) Int. Cl. A61K 36/746

(52) U.S. Cl. 424/769; 424/777

(57) ABSTRACT

The present invention is directed to methods and formulations for inhibiting Lipoxygenase or Lipoxygenase enzymes that function to biosynthesize or metabolize arachidonic acid into its intermediate Leukotriene constituents, as well as a method and formulation for treating and preventing inflammatory diseases and the symptoms associated with such diseases. The present invention methods and formulations effectively function as such through the introduction into the body (e.g. ingesting) a safe, pre-determined dosage of a naturaceutical composition formulated with or comprising one or more processed Morinda citrifolia products for a safe, pre-determined duration, wherein the processed Morinda citrifolia product may comprise one or more isolated active ingredients.

### BACKGROUND

1. Related Applications

This application claims priority to provisional application filed Mar. 26, 2003, for MORINDA CITRIFOLIA AS A 5-LIPOXYGENASE INHIBITOR.

2. Field of the Invention

The present invention relates to a composition formulated as an inhibitor of 5-Lipoxygenase, wherein the composition is formulated with one or more processed or unprocessed Morinda citrifolia ingredients or products as derived from the Indian Mulberry plant.

3. Background of the Invention and Related Art

Leukotrienes are a family of lipid mediators involved in acute and chronic inflammation and allergic response diseases. They are the biologically active metabolites of arachidonic acid and have been implicated in the pathological manifestations of inflammatory diseases, including asthma, arthritis, psoriasis, and inflammatory bowel disease. The biosynthesis of leukotrienes (LT or LT's) begins with the oxygenation of arachidonic acid into an unstable epoxide known as LTA₄ (an intermediate central to the formation of leukotrienes) by the enzyme 5-lipoxygenase (5-LO). LTA₄ can further be converted into the potent chemo attractant LTB₄ by the enzyme LTA₄ hydrolase or conjugated with glutathione (GSH) to produce LTC₄ by a specific microsomal GSH S-transferase (MGST) known as LTC₄ synthetase (LTC₄S). LTC₄ is the parent compound of the cysteinyl-leukotrienes (cys-LTs) that include LTC₄, LTD₄, and LTE₄. These three cysteinyl-leukotrienes are potent smooth muscle constricting agents, particularly in the respiratory and circulatory systems. These are mediated via at least two cell receptors, CysLT1 and CysLT2. The CysLT1 receptor is a G-protein-coupled receptor with seven transmembrane regions. There has been numerous amounts of data that has been collected, which clearly demonstrates that the CysLT's play a pivotal role in inflammatory and allergic response diseases, particularly asthma.

It has also been established that these lipid mediators have profound hemodynamic effects, constricting coronary blood vessels, resulting in a reduction of cardiac output efficiency. Moreover, CysLT's have been shown to induce secretion on von Willebrand factor and surface expression of P-selectin in cultured HUVEC. Von Willebrand is a genetic disorder. The most common types, and those most familiar to people, are the hemophiliac diseases. These enzymes of the 5-LO pathway produce active metabolites from arachidonic acid that cause inflammation. This has been shown both by the identification of higher levels of leukotrienes in both acute and chronic inflammatory lesions coupled with the evidence of primary signs of inflammation when leukotrienes are added to tissue cultures.

In addition, the cysteinyl LT's are predominantly secreted by eosinophils, mast cells, and macrophages, which cause vasodilatation, increase postcapillary venule permeability, and stimulate bronchoconstriction and mucous secretion. Furthermore, it has been observed that elevated leukotriene LTC₄ synthase activity was observed in peripheral blood granulocyte suspensions from patients with chronic myeloid leukemia (CML), and human bone marrow-derived myeloid progenitor cells. In asthma, the cysteinyl leukotrienes are present in alveolar lavage fluid of patients. Therefore, the presence of 5-LO and leukotriene synthase are clinically important in the diagnosis of patients with bronchial asthma.

Currently, in the market, two oral Leukotriene receptor antagonists used in the treatment of asthma are available. Release of leukotrienes within the body by an allergic reaction will promote inflammation in many diseases, such as asthma, a disease in which the inflammation occurs in the lungs.

### SUMMARY AND OBJECTS OF THE INVENTION

The present invention is directed to methods of and formulations for inhibiting 5-Lipoxygenase (5-LO) and the lipid mediators known as leukotrienes that contribute to the pathological manifestations of inflammatory diseases, namely, asthma, arthritis, psoriasis, and inflammatory bowel disease, as well as the treatment and prevention of these diseases through the introduction into the body (e.g. ingesting) a safe, pre-determined dosage of a composition formulated with or comprising one or more processed Morinda citrifolia ingredients or products for a safe, pre-determined duration. The present invention specifically functions to inhibit the oxygenation and metabolizing of arachidonic acid into its leukotriene synthesized intermediates by inhibiting the enzyme 5-LO that allows arachidonic acid to do so.

In one currently preferred embodiment, a quantity of a processed Morinda citrifolia product is obtained in the form of fruitjuice, puree juice or juice puree, pulp, seed oil, and/or dietary fiber, using the process(es) as described below. Subsequently, an amount of any one of or a combination of these is formulated with other ingredients to create a naturaceutical composition formulated to provide significant health advantages and to assist in the treatment of and provide preventative effects for inflammatory diseases through the inhibition of the 5-LO enzyme.

To achieve the foregoing objects, and in accordance with the invention as embodied and broadly described herein, the present invention features a neutraceutical composition formulated with at least one processed Morinda citrifolia product for the inhibition of 5-LO within the body of a mammal.

The present invention naturaceutical composition comprises at least one processed Morinda citrifolia product in one of its several forms (preferably the fruit juice), formulated with other ingredients, either natural or artificial, as needed. The preferred naturaceutical composition is a liquid that may be administered orally or through intravenous injection, wherein the active ingredients, namely Morinda citrifolia, are allowed to be absorbed into the tissues to inhibit Lipoxygenase and reduce/regulate leukotriene production.

The present invention further features a method of inhibiting 5-LO and treating, inhibiting, preventing, and reversing inflammatory diseases through the prophylactic administration of a naturaceutical composition comprising at least one processed Morinda citrifolia product as an active ingredient.

### DETAILED DESCRIPTION OF THE PREFERRED EMBODIMENTS

It will be readily understood that the components of the present invention, as generally described herein, could be arranged and designed in a wide variety of different configurations. Thus, the following more detailed description of the embodiments of the system and method of the present invention is not intended to limit the scope of the invention, as claimed, but is merely representative of the presently preferred embodiments of the invention.

The present invention describes and features a method and formulation for inhibiting 5-lipoxygenase (5-LO) and for treating and preventing inflammatory diseases within a mammal through the prophylactic administration of a naturaceutical formulation comprising at least one Morinda citrifolia product in processed form.

The presently preferred embodiments of the invention will be best understood, and its benefits and advantages more clearly pointed out, by separating the following more detailed description into sections, the first pertaining to a general discussion regarding Morinda citrifolia, including its origins, processing techniques, and health benefits, as well as the methods employed to produce and manufacture the processed Morinda citrifolia products used as key active ingredients in the naturaceutical formulations described herein; and the second being a more detailed and specific discussion on the naturaceutical formulations and compositions comprising the processed Morinda citrifolia product used to inhibit 5-LO and to treat and prevent inflammatory diseases. Examples of experimental studies and the results obtained are also provided herein.

### GENERAL DISCUSSION OF MORINDA CITRIFOLIA AND THE METHODS USED TO PRODUCE PROCESSED MORINDA CITRIFOLIA PRODUCTS

The Indian Mulberry plant, known scientifically as Morinda citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in the naturaceutical used to inhibit 5-LO and treat various inflammatory diseases, such as arthritis, asthma, etc. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process includes freeze-drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp a separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include, but are not limited to: from the leaves—alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers—acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; from the fruit—acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots—anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark—alizarin, chlororubin, glycosides (pentose, hexose), morindadiol, morindanigrine, morindine, morindone, resinous matter, rubiadin monomethyl ether, and soranjidiol; from the wood—anthragallol-2,3-dimethylether; (from the tissue culture): damnacanthal, lucidin, lucidin-3-primeveroside, and morindone-6beta-primeveroside; from the plant—alizarin, alizarin-alpha-methyl ether, anthraquinones, asperuloside, hexanoic acid, morindadiol, morindone, morindogenin, octanoic acid, and ursolic acid.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others. The compositions containing Morinda citrifolia may be in a form suitable for oral use, systemic administration, injection, and others. In regards to an oral composition, such a composition may exist, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method of inhibiting 5-LO with a Morinda citrifolia-based naturaceutical formulation without any significant tendency to cause gastric or other adverse side effects.

### MORINDA CITRIFOLIA-BASED NATURACEUTICAL FORMULATIONS AND METHODS OF ADMINISTRATION FOR INHIBITING 5-LIPOXYGENASE

The present invention advances treatment of inflammatory diseases and 5-LO enzyme inhibitors by providing a naturaceutical composition formulated with one or more processed Morinda citrifolia products derived from the Indian Mulberry plant. The Morinda citrifolia is incorporated into various carriers or naturaceutical compositions suitable for in vivo treatment of a patient. For instance, the inhibitor may be ingested, injected, introduced intravenously, or otherwise internalized as is appropriate and directed.

In one exemplary embodiment, the naturaceutical composition of the present invention comprises one or more of a processed Morinda citrifolia product present in an amount by weight between about 0.01 and 100 percent by weight, and preferably between 0.01 and 95 percent by weight. Several embodiment of formulations are provided below. However, these are only intended to be exemplary as one ordinarily skilled in the art will recognize other formulations or compositions comprising the processed Morinda citrifolia product. The processed Morinda citrifolia product comprises at least one of the active ingredients in the naturaceutical, or contains one or more active ingredients, such as Quercetin and Rutin, and others, for effectuating the inhibition of 5-LO and treating and preventing inflammatory diseases. The Morinda citrifolia product specifically functions to inhibit the oxygenation and metabolizing of arachidonic acid into its leukotriene synthesized intermediates or constituents. This is accomplished by inhibiting the enzyme 5-LO that functions as a catalyst allowing or facilitating arachidonic acid to synthesize to these intermediates.

Active ingredients within the processed Morinda citrifolia product may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The processed Morinda citrifolia product may be formulated with various other ingredients to produce various compositions, such as a naturaceutical composition, a topical dermal composition, or others. The ingredients to be utilized in a naturaceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the naturaceutical composition is preferably consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically (e.g. intravenously) administered formulations may also comprise any known in the art.

The present invention further features a method of administering a naturaceutical composition to a mammal to inhibit 5-LO, thus inhibiting the biosynthesis of leukotrienes, as well as to treat and prevent inflammatory diseases. In one exemplary embodiment, the method comprises the steps of (a) formulating a naturaceutical composition comprising in part a processed Morinda citrifolia product present in an amount between about 0.01 and 95 percent by weight, wherein the composition also comprises a carrier, such as water or purified water, and may also comprise other natural or artificial ingredients; (b) administering the naturaceutical composition into the body of a mammal, such that the processed Morinda citrifolia product is sufficiently internalized; (c) repeating the above steps as often as necessary to provide an effective amount of the processed Morinda citrifolia product to inhibit 5-LO therein.

The step of administering the naturaceutical composition into the body preferably comprises ingesting the composition orally through one of several means. Specifically, the naturaceutical composition may be formulated as a liquid, gel, solid, or some other type that would allow the composition to be quickly digested and concentrated within the colon. It is important to note that the step of administering the naturaceutical composition should be carried out in an effective manner so that the greatest concentration of naturaceutical composition is allowed to absorb into the tissues and cells. For the naturaceutical composition to take effect, it must be sufficiently internalized. Once sufficiently internalized, it may then begin to sufficiently inhibit or effectuate the inhibition of 5-LO, thus discouraging the oxygenation of arachidonic acid into its leukotriene constituents or active metabolites that adversely function within the inflammatory pathophysiology.

In another embodiment, the step of administering the naturaceutical composition may include injecting the composition into the body using an intravenous pump. This technique is advantageous as it would allow the composition to be localized in the area where it would have the most effect, or the area that would provide for the greatest concentration of the naturaceutical composition.

In one exemplary embodiment, the naturaceutical composition is administered by taking between 1 teaspoon and 2 oz., and preferably 2 oz., of the naturaceutical composition every two hours each day, or at least twice a day on a continued basis. Also, the naturaceutical composition is to be taken on an empty stomach, meaning at a period of time at least two hours prior to consumption of any food or drink. Following this, the naturaceutical composition resultantly functions to actively inhibit 5-LO. Of course, one ordinarily skilled in the art will recognize that the amount of composition and frequency of use may vary from individual to individual.

The following tables illustrate or represent some of the preferred formulations or compositions contemplated by the present invention. As stated, these are only intended as exemplary embodiments and are not to be construed as limiting in any way.

| | |
|---|---|
| Ingredients | Percent by Weight |

FORMULATION ONE

| | |
|---|---|
| Morinda citrifolia puree juice or fruit juice | 100% |

FORMULATION TWO

| | |
|---|---|
| Morinda citrifolia fruit juice | 85-99.99% |
| Water | 0.1-15% |

FORMULATION THREE

| | |
|---|---|
| Morinda citrifolia fruit juice | 85-99.99% |
| non-Morinda citrifolia-based fruit juices | 0.1-15% |

FORMULATION FOUR

| | |
|---|---|
| Morinda citrifolia fruit juice | 50-90% |
| Water | 0.1-50% |
| non-Morinda citrifolia-based fruit juices | 0.1-30% |

FORMULATION FIVE

| | |
|---|---|
| Morinda citrifolia puree juice | 85-99.9% |
| Water | 0.1-15% |

FORMULATION SIX

| | |
|---|---|
| Morinda citrifolia puree juice | 85-99.9% |
| non-Morinda citrifolia-based fruit juices | 0.1-15% |

FORMULATION SEVEN

| | |
|---|---|
| Morinda citrifolia puree juice | 50-90% |
| Water | 0.1-50% |
| non-Morinda citrifolia-based fruit juices | 0.1-30% |

FORMULATION EIGHT

| | |
|---|---|
| Morinda citrifolia dietary fiber | 0.1-30% |
| Water | 1-99.9% |
| non-Morinda citrifolia-based fruit juices | 1-99.9% |

FORMULATION NINE

| | |
|---|---|
| Morinda citrifolia dietary fiber | 0.1-30% |
| Water | 1-99.9% |
| Morinda citrifolia fruit juice or puree juice | 1-99.9% |

FORMULATION TEN

| | |
|---|---|
| Morinda citrifolia oil | 0.1-30% |
| carrier medium | 70-99.9% |
| other ingredients | 1-95% |

FORMULATION ELEVEN

| | |
|---|---|
| Morinda citrifolia product | 10-80% |
| carrier medium | 20-90% |

FORMULATION TWELVE

| | |
|---|---|
| Morinda citrifolia product | 5-80% |
| carrier medium | 20-95% |

FORMULATION THIRTEEN

| | |
|---|---|
| Morinda citrifolia oil or oil extract | 0.1-20% |
| carrier medium | 20-90% |

FORMULATION FOURTEEN

| | |
|---|---|
| Morinda citrifolia puree juice or fruit Juice | 0.1-80% |
| Morinda citrifolia oil | 0.1-20% |
| carrier medium | 20-90% |

FORMULATION FIFTEEN

| | |
|---|---|
| Morinda citrifolia puree juice concentrate or fruit | 100% |
| juice concentrate | |

FORMULATION SIXTEEN

| | |
|---|---|
| Morinda citrifolia fruit juice concentrate or puree | 85-99.99% |
| juice concentrate | |
| Water | 0.1-15% |

As stated, in one exemplary embodiment, the present invention features a method for introducing an internal composition or formulation to inhibit 5-LO and to inhibit the oxygenation of arachodonic acid into its leukotriene intermediate constituents, for the purpose of treating and preventing inflammatory diseases. This method essentially comprises the introduction of an internal composition into the body of a mammal suffering from one of such diseases. Several embodiments of the internal composition comprising various different ingredients are contemplated for use herein, with each embodiment comprising one or more forms of a processed Morinda citrifolia product as taught and explained herein and a carrier agent or medium.

In one preferred method, 5-LO is inhibited, and an inflammatory disease is treated, prevented, destroyed, and/or reversed, by administering at least one (1) ounce of one of Formulations One through Sixteen above in the morning on an empty stomach, and at least one (1) ounce at night on an empty stomach, just prior to retiring to bed. In one example, which is not meant to be limiting in any way, the beneficial Morinda citrifolia is processed into Tahitian Noni® juice manufactured by Tahitian Noni International of Provo, Utah.

In one exemplary embodiment, the internal composition comprises the ingredients of: a processed Morinda citrifolia product present in an amount by weight between about 10-80 percent; and a carrier medium present in an amount by weight between about 20-90 percent.

In this embodiment, the processed Morinda citrifolia product may comprise one or more of a processed Morinda citrifolia fruit juice, processed Morinda citrifolia puree juice, processed Morinda citrifolia dietary fiber, and/or processed Morinda citrifolia oil extract product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

According to the present invention, the particular methods of introducing an internal composition may comprises any method of actually introducing the internal composition into the body of a mammal for the purposes identified herein. Although the particular methods are many, the present invention recognizes that the internal composition may be introduced intravenously, transdermally, orally, or systemically. No matter what method is employed, it is important to thoroughly internalize the composition so that the internal composition, and particularly the Morinda citrifolia and other active ingredients, can effectively inhibit 5-LO and treat any inflammatory diseases.

The carrier medium identified in the above Formulations may comprise any ingredient capable of being introduced into the body of a mammal, and that is also capable of providing the carrying medium to the processed Morinda citrifolia product. Specific carrier mediums formulations are well known in the art and not described in detail herein. The purpose of the carrier medium is as stated, to provide a means to embody the processed Morinda citrifolia product within the internal composition that is capable of being introduced into the body.

The following examples set forth and present the preventative and treatment effects of the processed Morinda citrifolia products on 5-LO, as well as the preventative and treatment effects of Morinda citrifolia against inflammatory diseases. These examples are not intended to be limiting in any way, but are merely illustrative of the benefits and advantageous, as well as the remedial effects, of the Morinda citrifolia products.

### Example One

There are several main branches of the arachidonic acid cascade that affect inflammation and drug metabolism. Specifically, a first branch of the arachidonic acid cascade results in synthesis by several Cytochrome (CYP) enzymes, namely CYP450, CYP2D6, CYP2C19, CYP3A4, CYP 2C9, and CYP1A2. The second branch is the arachidonic acid cascade into cyclooxygenase-1 and 2 (COX 1 and COX 2). The third branch of the arachidonic acid cascade involves 5-lipoxygenase pathways and its enzymes, such as Leukotriene A4 (LTA4) hydrolase and Leukotriene C₄ (LTC₄) synthetase.

Lipoxygenase is required for development of senescence and production of antibacterial compounds in plants. In mammals, lipoxygenase catalyzes the oxidation of arachidonic acid into Eicosanoic acids (5, 12, and 15). Lipoxygenase are enzymes that contain nonheme iron and that use molecular oxygen in the metabolism of arachidonic acid, and also facilitates the biosynthesis of potent mediators, leukotrienes, that have far reaching physiological effects in mammals.

Inflammatory and allergic responses are modulated by arachodonic acid metabolites through a variety of interconnected mechanisms. Important signal molecules (LT's, PG's) in a variety of pathways of inflammation and allergic conditions affect the skin, joints, gastrointestinal, and respiratory systems, in particular asthma. Arachodonic acid is metabolized into various active leukotrienes, namely LTD₄, LTB₄, etc. These are potent inflammatory mediators that contribute to increased mucus production, bronchoconstriction, and eisoniphil infiltration. It has also been shown that products of 5-LO activities are significant activators of microglia.

The central nervous system comprises neurons and glial cells. Glial cells are supporting cells that provide nutrients and oxygen, insulate one neuron from another, surround and hold neurons in place, and destroy and remove dead neurons. Microglia are the smallest of the glial cells. Microglia become reservoirs for infectious agents, viruses, fungi, etc, which contribute to inflammation. As infected cells in the central nervous system, microglia play a key role in development of various diseases, possibly by the production of toxic factors following infection, or more directly by not providing normal metabolic support for neurons. Moreover, neuroinflammation and oxidative stress are believed to be contributing factors to neurodegeneration in normal aging and in age-related neurological disorders, such as AD, PD, ALS, and Osteoarthritis. Reactive microglia are found in increasing numbers compared to normal tissues. In vitro, stimulated microglia or microglia-like cells secrete neurotoxic materials, and are generators of free radicals through the respiratory burst system. This has led to the hypothesis that activation of these cells could contribute to the death of neuron cells observed in diseased brain tissue. Reactive microglia are round in increasing numbers in the aging brain. They generate large amounts of toxic oxygen free radicals. Any agents that suppress microglia activation are good candidates for protection of neurons.

The present experiment involved the dual inhibition of both cyclooxygenase-1 and 2 and 5-lipoxygenase. The COX pathway generates inflammatory PG's, while the 5-LO pathway generates inflammatory leukotrienes. Inhibitors of both pathways suppressed neurotoxicity in a dose dependent manner. Both pathway inhibitors are more effective than a single pathway inhibitor as dual COX and 5-LO pathway inhibitors reduce the level of cartilage chondrocyte death.

In this study, the 5-LO source was human peripheral blood mononuclear cells (PBMNC) and the substrate was arachidonic acid having a general reaction of: arachidonic acid→5-LO→5-HPETE→LTB₄. The inhibiting agent was processed Morinda citrifolia puree juice concentrate. The results of the study indicated that processed Morinda citrifolia puree product, as described and taught herein, functioned as a significant inhibitor. In regards to the 5-LO pathways, the Morinda citrifolia puree functioned to inhibit the 5-LO enzyme, which resulted in the inhibition of the oxygenation of arachidonic acid into its intermediate leukotriene constituents, such as LTA₄ hydrolase, LTA₄, and LTC₄ synthetase. In one specific study, introduction of a one percent (1%) Morinda citrifolia puree juice concentrate provided a one hundred and three percent (103%) inhibition of 5-LO enzyme.

Based on these results, it appears that the synergistic inhibition of 5-LO using one or more Morinda citrifolia products provides a promising way to treat and prevent inflammatory diseases with little or no stomach irritation by lowering or inhibiting the production of inflammatory mediators, such as LTA₄, LTB₄, LTC₄, etc. As such, it is proposed that the Morinda citrifolia products described herein possess significant anti-inflammatory properties.

### Example Two

In another example, one liter of processed Morinda citrifolia puree juice was placed in a centrifuge and mixed with one liter of 200 Proof Ethanol for the purpose of obtaining an alcohol supernate. The mixture was allowed to mix for three hours in the centrifuge to remove the precipitate from the alcohol supernate. The precipitate was further used in the isolation of the PPT. The alcohol supernate was collected (alcohol soluble fraction) and the alcohol was then removed using the RotoVap with pressure until a brown syrup-like substance was obtained. A small sample was ran on the HPLC to make sure no alcohol remained, and none was detected.

From this, using the same assay and protocols as outlined in Example One, it was discovered that the alcohol supernate fraction provided one hundred and two percent (102%) inhibition of the 5-LO enzyme.

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1-16. (canceled)

17. A method for inhibiting Lipoxygenase and for treating and preventing inflammatory diseases within a mammal, said method comprising the steps of:
adding a processed Morinda citrifolia product to an alcohol-based solution;
isolating and extracting an active ingredient of said processed Morinda citrifolia product from said solution to obtain a fraction;
introducing said extracted active ingredient into said mammal, wherein said extracted active ingredient inhibits, prevents, reduces, and reverses the effects of inflammatory diseases.

18. The method of claim 17, wherein said processed Morinda citrifolia product comprises processed Morinda citrifolia fruit juice.

19. The method of claim 17, wherein said processed Morinda citrifolia product comprises processed Morinda citrifolia puree.

20. The method of claim 17, wherein said alcohol-based solution is selected from the group consisting essentially of methanol, ethanol, and ethyl acetate, and other alcohol-based derivatives.

21. The method of claim 17, wherein said active ingredient is Quercetin.

22. The method of claim 21, wherein said active ingredient is Rutin that synergistically works with said Quercetin to inhibit said Lipoxygenase.

23. The method of claim 17, wherein said active ingredient is a soluble alcohol supernate fraction.

24. The method of claim 17, wherein said step of introducing said extracted active ingredient comprises orally administering two ounces of a food product to a patient twice daily each day on an empty stomach, said food product comprising said active ingredient obtained from said processed Morinda citrifolia product.

25. The method of claim 17, wherein said step of introducing said extracted active ingredient comprises applying a topical composition to said area, said topical composition comprising said active ingredient obtained from said processed Morinda citrifolia product.

26. A method for inhibiting Lipoxygenase and treating and preventing inflammatory diseases and the symptoms associated therewith within a mammal, said method comprising the steps of:
orally administering to said mammal at least one ounce of a naturaceutical formulation comprising a processed Morinda citrifolia product on an empty stomach in the morning;
orally administering to said mammal at least one ounce of said naturaceutical formulation on an empty stomach prior to sleeping at night; and
repeating said steps of orally administering each day until said estrogen-dependent cancerous cells are destroyed.

27. The method of claim 26, wherein said naturaceutical formulation comprises:
processed Morinda citrifolia fruit juice present in an amount by weight of about 100 percent.

28. The method of claim 26, wherein said naturaceutical formulation comprises:
processed Morinda citrifolia fruit juice present in an amount by weight between about 85-99.99 percent; and
water present in an amount by weight between about 0.1-15 percent.

29. The method of claim 26, wherein said naturaceutical formulation comprises:
processed Morinda citrifolia fruit juice present in an amount by weight between about 85-99.99 percent; and
non-Morinda citrifolia-based fruit juices present in an amount by weight between about 0.1-15 percent.

30. The method of claim 26, wherein said naturaceutical formulation comprises:
processed Morinda citrifolia fruit juice present in an amount by weight between about 50-90 percent;
water present in an amount by weight between about 0.1-50 percent; and
non-Morinda citrifolia-based fruit juices present in an amount between about 0. 1-30 percent.

31. The method of claim 26, wherein said naturaceutical formulation comprises:
processed Morinda citrifolia puree juice present in an amount by weight of about 100 percent.

32. The method of claim 26, wherein said naturaceutical formulation comprises:
processed Morinda citrifolia puree juice present in an amount by weight between about 85-99.99 percent; and
water present in an amount by weight between about 0.1-15 percent.

33. The method of claim 26, wherein said naturaceutical formulation comprises:
processed Morinda citrifolia puree juice present in an amount by weight between about 85-99.99 percent; and
non-Morinda citrifolia-based fruit juices present in an amount by weight between about 0.1-15 percent.

34. The method of claim 26, wherein said naturaceutical formulation comprises:
processed Morinda citrifolia puree juice present in an amount by weight between about 50-90 percent;
water present in an amount by weight between about 0.1-50 percent; and
non-Morinda citrifolia-based fruit juices present in an amount between about 0. 1-30 percent.

35. The method of claim 26, wherein said step of orally administering comprises orally administering two ounces of said naturaceutical formulation.

36-62. (canceled)
